(19) United States
(12) Patent Application Publication
(10) Pub. No.: US 2008/0243135 A1
(43) Pub. Date: Oct. 2, 2008

(54) Driver-Fixator System, Method, and Apparatus

(76) Inventor: Randolph C. Robinson, Centennial, CO (US)

Correspondence Address: Neugeboren O''Dowd PC, 1227 Spruce Street, SUITE 200, BOULDER, CO, 80302, US

(21) Appl. No.: 11/969,915

(22) Filed: Jan. 6, 2008

Related U.S. Application Data
(60) Provisional application No. 60/909,320, filed on Mar. 30, 2007.

(51) Int. Cl. A61B 17/58; B25B 13/00

(52) U.S. Cl. 606/104; 81/52

(57) ABSTRACT

The invention may be characterized as a method, apparatus and bone-fixation system. The system includes a plurality of drivers, each of the drivers including a handle coupled to a shaft, the shaft including a distal tip opposite the handle that is configured to couple with a head of a surgical fixation element, each of the drivers including indicia of a particular length and a particular diameter; and a plurality of surgical fixation elements, each of the plurality of surgical fixation elements being detachably coupled to the distal tip of a corresponding one of the drivers that has indicia of the length and diameter of the fixation element coupled to the driver.

### PRIORITY

The present application claims priority to provisional patent application No. 60/909,320, filed Mar. 30, 2007, entitled: Bone Screw/Screwdriver System and Method, which is incorporated by reference herein in its entirety.

### FIELD OF THE INVENTION

This invention relates generally to surgical systems, methods, and apparatus, and in particular, to bone fixation systems, methods, and apparatus.

### BACKGROUND OF THE INVENTION

During various surgical procedures it is necessary to insert bone screws to hold bones and bone plates in position. Typically, each screw is loaded separately onto a screwdriver and then passed to the surgeon after the surgeon communicates the proper size of the screw to the assistant. The assistant then passes the loaded screwdriver to the surgeon for insertion. This process is repeated many times until the bone fixation is complete.

It is difficult to identify the proper screw and then insert the tip of the screwdriver into the screw—especially in the context of micro screw systems. And once the screwdriver is inserted into the screw, the screw may fall off the screwdriver, become lost on the floor of the surgical field, or the wrong size may have been selected. As a consequence, surgical procedures are often hindered by the arduous and frustrating process of loading and passing the bone screw/screwdriver to the surgeon. Accordingly, a system and method are needed to address the shortfalls of present technology and to provide other new and innovative features.

### SUMMARY OF THE INVENTION

In one embodiment, the invention may be characterized as a system, the system including a plurality of drivers. Each of the drivers in this embodiment including a handle coupled to a shaft that includes a distal tip opposite the handle that is configured to couple with a head of a surgical fixation element, and each of the drivers includes indicia of a particular length and a particular diameter. The system in this embodiment additionally includes a plurality of surgical fixation elements that are detachably coupled to the distal tip of a corresponding one of the drivers that has indicia of the length and diameter of the screw coupled to the driver.

In accordance with another embodiment, the invention may be characterized as a method. The method in this embodiment includes collecting a plurality of drivers, each of the drivers including a handle coupled to a shaft, the shaft including a distal tip opposite the handle that is configured to couple with a head of a surgical screw. Each of the drivers in this embodiment includes indicia of a particular screw length, and each of the plurality of surgical screws is coupled to the distal tip of a corresponding one of the drivers that has indicia of the length of the screw. Each of the collection of the plurality of drivers may be placed in a storage unit configured to hold the plurality of drivers, and then the collection of the plurality of drivers may be sent to another physical location while the plurality of drivers are coupled to the plurality of surgical screws.

In accordance with yet another embodiment, the invention may be characterized as a method. The method in this embodiment includes receiving a plurality of driver-fixator assemblies, each of the driver-fixator assemblies including a handle, a shaft and a fixation-element. The handle in this embodiment is coupled to the shaft that includes, opposite the handle, a distal tip that is coupled with the fixation-element, and each of the drivers includes indicia of a size of the fixation-element that is coupled to the shaft. One of the plurality of driver-fixator assemblies is retrieved and the fixation-element that is coupled to the retrieved driver-fixator assembly is driven into a bone.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings wherein:

FIG. 1 is a perspective view of an exemplary embodiment of a driver-fixator assembly;

FIG. 2 is a perspective view of an exemplary embodiment of a driver-fixator storage unit, which may be used to store the driver-fixator assembly depicted in FIG. 1;

FIG. 3 is a perspective view of another exemplary embodiment of a driver-fixator storage unit; which may be used to store the driver-fixator assembly depicted in FIG. 1;

FIG. 4 is a perspective view of a storage system that may be used in connection with the embodiments described with reference to FIGS. 1-3;

FIG. 5 is a block diagram depicting a portion of an exemplary system, which may be used to distribute driver-fixator assemblies; and

FIG. 6 is a flowchart depicting an exemplary method for distributing driver-fixator assemblies.

### DETAILED DESCRIPTION

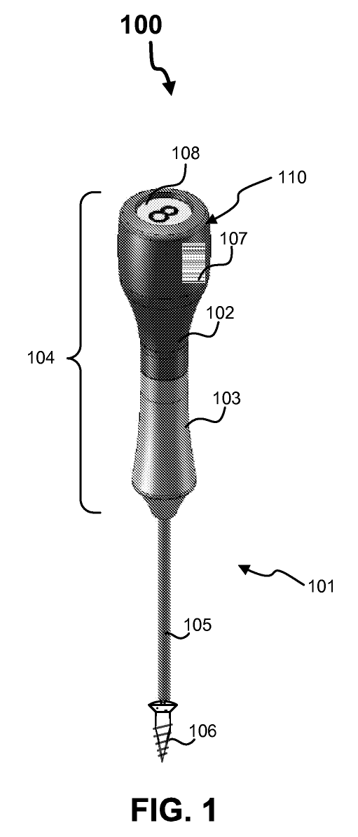

Referring first to FIG. 1, shown is a perspective view of a driver-fixator assembly 100. As shown, the exemplary driver-fixator assembly 100 includes a fixation element 106, depicted for example as a screw, that is securely premounted (e.g., friction mounted) onto a shaft 105 of a driver portion 101. As depicted, the driver portion 101 also includes a handle portion 104 that includes an intermediate segment 103 and a swivel segment 102, but this is certainly not required, and in other embodiments the handle portion 104 may be a unitary component. Also shown are a first size indicia 108 and a second size indicia 110, which include indicia of the length and diameter of the fixation element 106. Although not required, the first size indicia may be a numeral indicative of a length of the fixation element 106, and the second size indicia 110 may be a color that is indicative of a diameter of the fixation element.

Beneficially, the fixation-element 106 is coupled to a tip of the shaft 105 so that it will not fall off until the user (e.g., surgeon and/or surgical assistant) desires to release the fixation-element 106 from the driver 101. In one implementation for example, the fixation-element 106 and driver 101 are configured so that the fixation-element 106 is held in place at the tip of the driver 101 by just enough force to prevent the fixation-element 106 from falling off the driver 101 in advance of the fixation-element 106 being engaged in a desired location (e.g., within a patient's bone). And once the fixation-element 106 is engaged, the user (e.g., surgeon) simply pulls the driver 101 away from the fixation-element 106 to release the driver 101 from the fixation-element 106.

As discussed further herein, the driver-fixator assembly 100 and systems incorporating the driver-fixator assembly 100 will enable surgical procedures to be carried out much more efficiently, more quickly, and with less frustration because the fixation-element 106 (e.g., a screw) is preloaded on the driver 101 so that there will be less screw loss from spillage and mishandling; thus saving money. Moreover, the indicia 108, 110 on the exemplary driver 101 enables surgical-room personnel to quickly and accurately identify both the fixation-element length and fixation-element diameter. As a consequence, procedures will be safer for the patient because the flow and communication in the operating room will improve, and because the likelihood of infection will be reduced due to more expeditious procedures.

Although the driver 101 is depicted in FIG. 1 with a numeral identifying a length of the fixation-element 106 and a color identifying a diameter of the fixation element, it is certainly contemplated that other schemes may be utilized to convey the size of the fixation-element 106 to a user of the assembly 100. For example, other physical aspects (e.g., length, topology, width) of the driver 101 may vary according the length and diameter of the screw.

As depicted, the driver 101 in this embodiment also includes machine readable information 107, depicted for example as a bar code. Advantageously, the machine readable information 107 enables a circulating nurse to easily log and register the fixation-elements because once a fixation-element from a driver-fixator assembly is used, the remaining driver may scanned (e.g., by a bar code reader) to identify and keep a record of the type of fixation-element was used during the procedure. Although a bar code is utilized in this embodiment, it is certainly contemplated that other technologies (e.g., RFID technology) may be utilized to scan drivers 101 after use. In yet other embodiments, drivers 101 do not include machine readable information, and yet, the indicia 108, 110 on the drivers enables the circulating nurse to identify the type of fixation-element used during the procedure.

In several embodiments, the fixation-element 106 that the driver 101 is configured to couple to is a surgical screw. But variations of the driver 101 may be used in connection with other types of fixation-elements. For example, rivets, staples, wires, pins, traction screws, and nails may also be used.

The shaft 105 may be made of surgical stainless steel, and the handle may be made from metals (e.g., aluminum) or a variety of plastics (e.g., high molecular weight plastic) that may, but need not, be injection molded. In many embodiments the driver 101 is designed and constructed to be disposable so that once a driver-fixator assembly 100 is used, the driver 101 is simply disposed of. In other embodiments, the driver 101 is designed and configured to be reused one or more times.

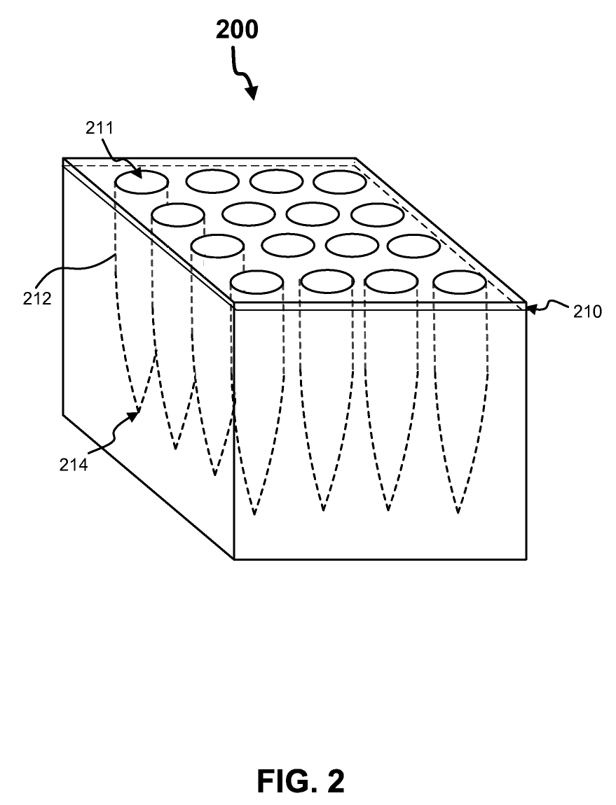

Referring next to FIG. 2, shown is a perspective view of a storage unit 200, also referred to herein as a retainer portion, which may be used to store driver-fixator assemblies (e.g., driver-fixator assembly 100). As shown, each holding slot 212, also referred to as a sleeve, is tapered from an opening 211 of the holding slot 212 to a sealed end 214. In addition, a retention flange 210 is included and disposed so as to rest over the openings 211 of the slots 212. In this way, when driver-fixator assemblies are placed into the slots 212, the driver-fixator assemblies are contained between the sealed end 214 of the slots and the retention flange 210 so as to prevent each driver (e.g., driver 101) from being uncoupled from its corresponding fixation element (e.g., fixation-element 106).

As a consequence, driver-fixator assemblies may be placed into the storage unit 200 at one physical location (e.g., a location where each driver is coupled to a fixation-element) and shipped to another remote location (e.g., a surgical facility) while each of the drivers is coupled to a corresponding one of the fixation elements. And when the storage unit 200 arrives at its destination, the retention flange 210 is simply removed and the driver-fixator assemblies are ready for use without the awkward and time consuming process of sequentially loading fixation-element after fixation-element on to a driver during a surgical procedure.

In some embodiments, each of the holding slots 212 may be sized according to the length of the particular driver and/or fixation-element. Moreover, the slots 212 may be configured so that the handle of each driver extends outside of the slot opening 211 so that the handles of the drivers can be readily grasped without displacing the screws from the tip of the screwdriver.

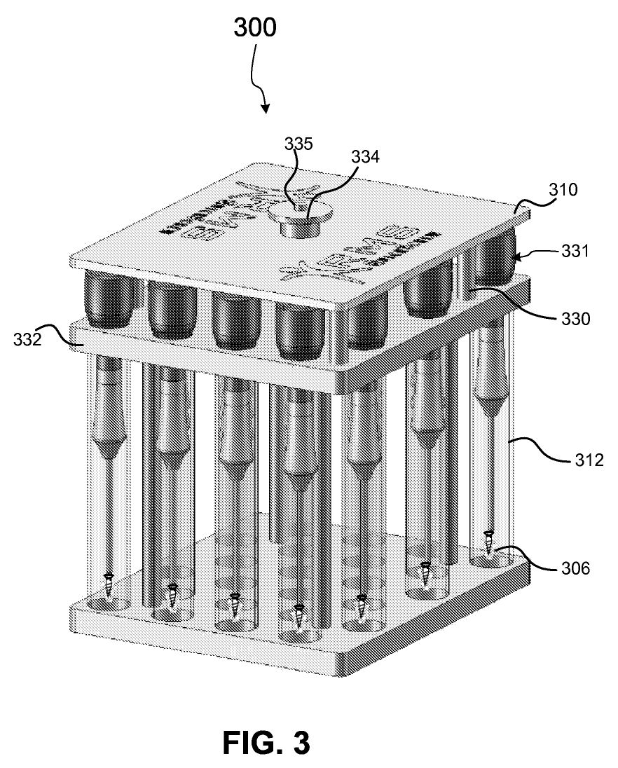

Referring next to FIG. 3 for example, shown is a perspective view depicting another embodiment of a storage unit 300. As shown, in this embodiment a retention flange 310 is raised with pedestals 330 above a slot-opening-platform 332 so that the handles 331 of the driver-fixator assemblies sit above the slot-opening platform 332; thus when the retention flange 310 is removed, the handles 331 of the drivers may be easily grasped while the handles 331 are sitting in the storage unit 300. As shown, the retention flange 310 in this embodiment is removably coupled to the slot-opening platform 332 with an expanding ring 334 that may be released by pushing button 335.

Although not required, the slots 312 in this embodiment are configured to enable driver-fixator assemblies of a variety of sizes to fit within each of the slots 312. In particular, the slots 312 depicted in the storage unit 300 are not tapered so that the fixation-elements 306 do not come in contact of a bottom portion of the slots 312. In this embodiment, the fixation-elements 306 may stay coupled to the drivers by friction coupling during transportation of the storage unit 300 from one location to another location.

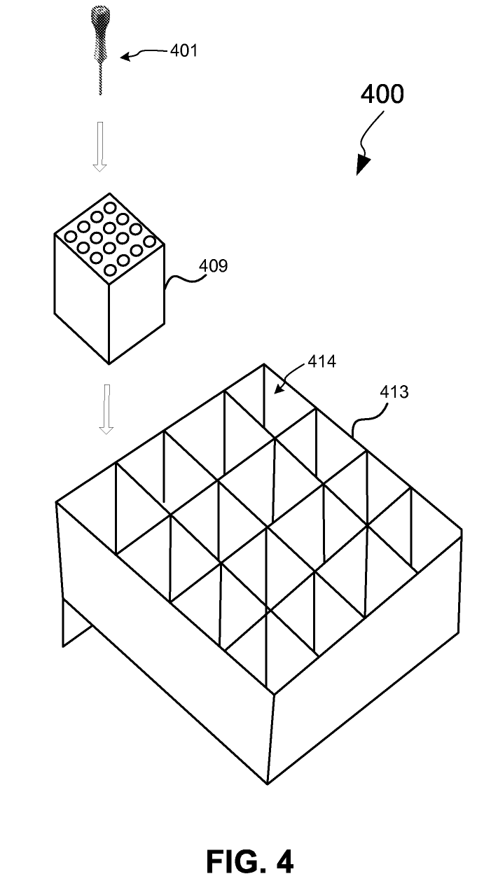

Referring next to FIG. 4, shown is a storage system 400 that may be used in connection with the embodiments described with reference to FIGS. 1-3. As depicted, each storage unit 409 (e.g., storage units 200, 300) may be removably stored in one of multiple compartments 414 in the holding tray 413 so that if desired, the storage units 409 may be removed from the holding tray 413 and organized in a desired manner. Beneficially, the embodiment depicted in FIG. 4 enables a broad array of fixation-element sizes to be easily distinguished, retrieved and utilized without the time consuming and error-prone process of manually loading fixation-elements on to ordinary drivers.

In one embodiment for example, each of the driver-fixator assemblies in each of the storage units 409 may include fixation-elements of one size so that storage units 409 may be organized within the holding tray 413 by the size of the fixation-element.

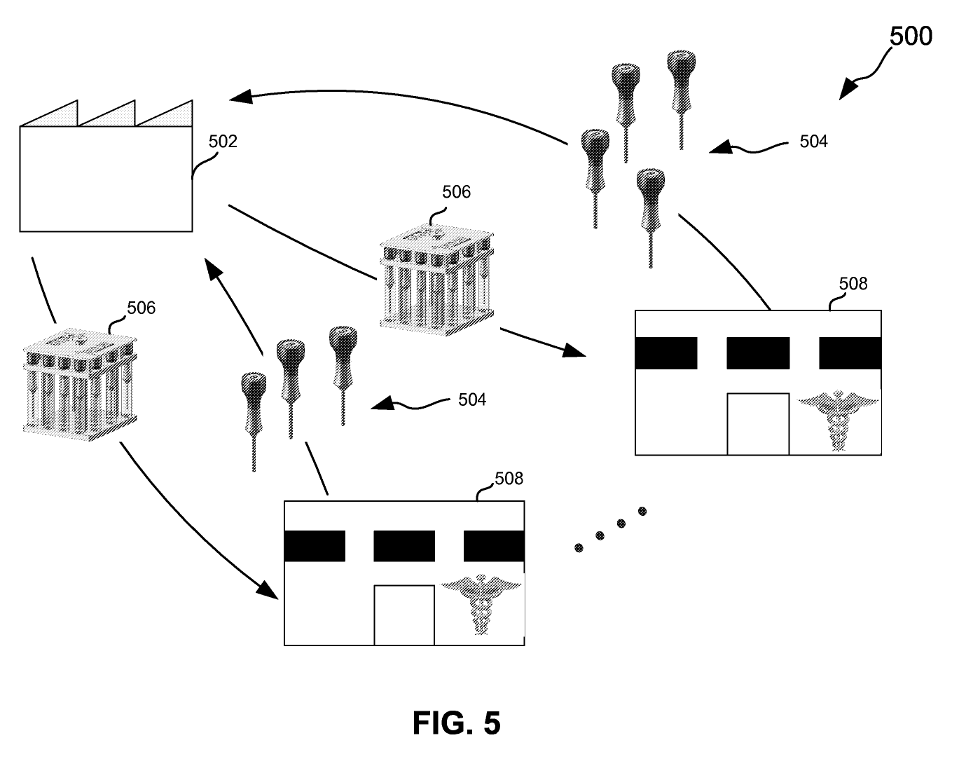

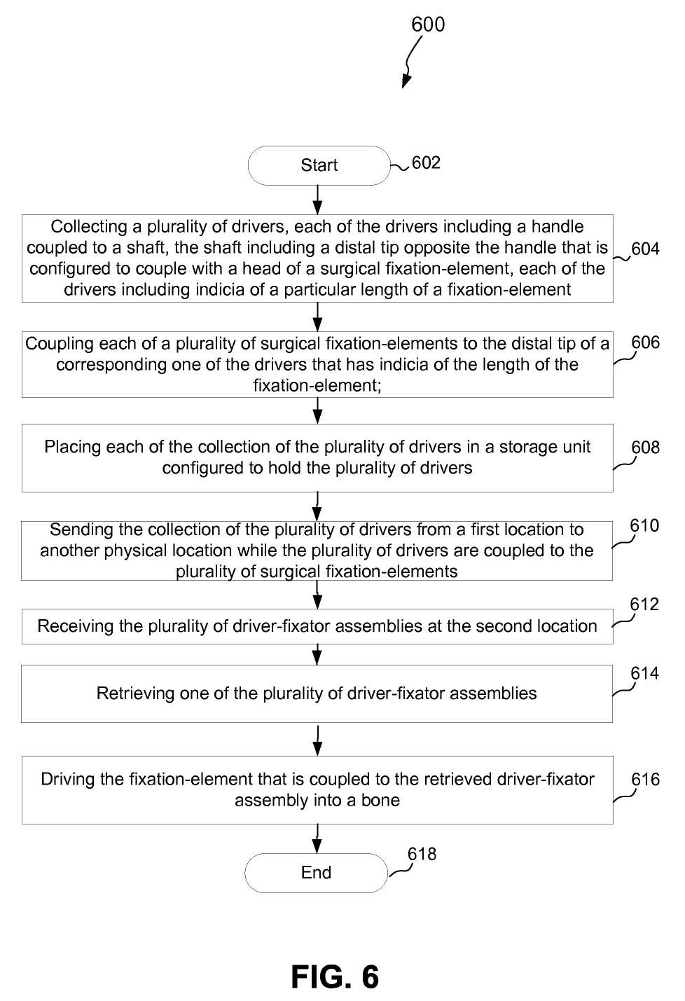

Referring next to FIG. 5 shown is a schematic representation of aspects of an exemplary system 500 in accordance with many embodiments of the present invention. While referring to FIG. 5, simultaneous reference will be made to FIG. 6, which is a flowchart 600 depicting an exemplary method for distributing driver-fixator assemblies. It should be recognized, however, that reference is made to both FIGS. 5 and 6 merely for exemplary purposes and the system 500 is not limited to the procedure outlined in the flowchart 600. Similarly, the method depicted in FIG. 6 is not limited to implementation within the exemplary system 500 depicted in FIG. 5.

As shown, a plurality of drivers 504 are collected at a first location (e.g., an assembly plant 502) (Blocks 602, 604), and each of a plurality of fixation-elements is coupled to a corresponding one of the drivers (Block 606). In several embodiments, each of the drivers (e.g. driver 101) includes indicia to particularly identify a length of a fixation element (e.g. fixation-element 106), and in these embodiments, fixation-elements are coupled to the drivers that identify the lengths of the corresponding fixation-elements to form a plurality of driver-fixator assemblies (e.g., driver-fixator assembly 100). And in many embodiments, the drivers may include both, indicia to identify a particular length and indicia to identify a particular diameter of fixation-element. In this way, each driver may immediately convey to a user (e.g., surgeon or surgical assistant) the length and diameter of the fixation-element that is coupled to the driver.

As depicted, in many embodiments each of the collection of the plurality of drivers is placed in a storage unit 506 (e.g., storage units 200, 300) that is configured to hold the plurality of drivers (Block 608), and the collection of the plurality of drivers is sent from a first location 502 to another physical location 508 while the plurality of drivers are coupled to the plurality of surgical fixation-elements (e.g., the drivers and the fixation elements are shipped coupled together as components of driver-fixator assemblies) (Block 610). The corporate entity operating the assembly at the first location 502 may or may not be affiliated with the operators of the remote facilities 508. It is also contemplated that each of the remote locations 508 may be affiliated or unaffiliated entities and may be physically dispersed at different locations around the earth.

Once the plurality of driver-fixator assemblies are received at the second location 508 (Block 612), each of the driver-fixator assemblies are ready to be utilized so that when a driver-fixator assembly is retrieved (Block 614), the fixation element is already coupled to the driver, and the surgeon may simply drive the fixation-element into a bone of the patient (Blocks 616, 618).

Once a fixation-element is inserted (e.g., into a bone) and the driver released, it may then passed off the surgical field for registry, tracking and billing. Beneficially, in many embodiments each driver that has been used indicates the type (e.g., length and/or diameter) of the fixation-element that was used in surgery so that an inventory of drivers also provides an accounting of the fixation-elements used during the procedure.

In many embodiments, once drivers are used, they are sterilized (e.g., by autoclaving procedures) at the first location 502 or other facility, and then fixation-elements are coupled to the drivers that identify the corresponding fixation element. The driver-fixator assemblies may then again be placed in a storage unit 506 and sent out to remote surgical facilities 508.

In other embodiments, drivers 504 are designed and constructed so as to be disposable so that used drivers are properly disposed of and the drivers received at the first location 502 are unused (e.g., received from a supplier of disposable drivers).

In conclusion, the present invention provides, among other things, a system, apparatus and method for rendering surgical bone fixation procedures more efficient and safe. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

What is claimed is:

1. A system including:
a plurality of drivers, each of the drivers including a handle coupled to a shaft, the shaft including a distal tip opposite the handle that is configured to couple with a head of a surgical screw, each of the drivers including indicia of a particular screw length and diameter;
a plurality of surgical screws, each of the plurality of surgical screws being detachably coupled to the distal tip of a corresponding one of the drivers that has indicia of the length and diameter of the screw coupled to the driver; and
a storage unit configured to hold the plurality of drivers so as to enable the plurality of drivers to be moved from one physical location to another physical location while the plurality of drivers are coupled to the plurality of surgical screws.

2. The surgical system of claim 1, wherein the indicia includes a color indicative of the particular screw diameter and a number indicative of the particular screw length.

3. The surgical system of claim 1, wherein the each of the plurality of screws is coupled, by friction, to the distal tip of the corresponding one of the drivers.

4. The surgical system of claim 1, wherein the plurality of drivers are selected, based upon screw length and diameter, for use during a particular surgical procedure.

5. The surgical system of claim 1, wherein the plurality of drivers are designed and include materials so as to enable the plurality of drivers to be sterilized and reused.

6. The surgical system of claim 1, wherein the each of the drivers includes machine-scannable information that identifies each driver.

7. A method comprising:
collecting a plurality of drivers, each of the drivers including a handle coupled to a shaft, the shaft including a distal tip opposite the handle that is configured to couple with a head of a surgical screw, each of the drivers including indicia of a particular screw length;
coupling each of a plurality of surgical screws to the distal tip of a corresponding one of the drivers that has indicia of the length of the screw;
placing each of the collection of the plurality of drivers in a storage unit configured to hold the plurality of drivers; and
sending the collection of the plurality of drivers to another physical location while the plurality of drivers are coupled to the plurality of surgical screws.

8. The method of claim 7, including:
placing each of the collection of the plurality of drivers in the storage unit after the plurality of surgical screws are coupled to the distal tip of a corresponding one of the drivers.

9. The method of claim 7, including:
receiving the plurality of drivers, each of the plurality of drivers having been previously used during a surgical procedure; and
sterilizing the collection of the plurality of drivers before sending the collection of the plurality of drivers to the other physical location.

10. The method of claim 7, including placing each of the collection of the plurality of drivers in a storage unit configured to hold the plurality of drivers for a particular surgical procedure.

11. The method of claim 7, wherein each of the drivers includes indicia of a particular screw diameter.

12. A method comprising:
receiving a plurality of driver-fixator assemblies, each of the driver-fixator assemblies including a handle, a shaft and a fixation-element, the handle coupled to the shaft, the shaft including, opposite the handle, a distal tip that is coupled with the fixation-element, each of the drivers including indicia of a size of the fixation-element that is coupled to the shaft;
retrieving one of the plurality of driver-fixator assemblies; and
driving the fixation-element that is coupled to the retrieved driver-fixator assembly into a bone.

13. The method of claim 12, wherein the indicia of the size of the fixation-element includes indicia of a length of the fixation-element.

14. The method of claim 12, wherein the fixation-element is a screw.

15. The method of claim 12, including scanning machine readable information on the handle of the retrieved driver-fixator assembly to track a use of the driver-fixator assembly.

16. The method of claim 12, including:
discarding the handle and shaft of after driving the fixation-element into the bone.

17. The method of claim 12, including:
gathering used handles and shafts; and
sending the used handles and shafts to a remote physical location for reprocessing.

18. A system comprising:
a retainer portion, the retainer portion including a plurality of apertures, each of the apertures configured to hold a corresponding one of a plurality of driver-fixator assemblies, each of the driver-fixator assemblies including a driver and a fixation element, the driver including a handle and a shaft, the fixation element coupled to a distal end of the shaft opposite the handle; and
a plurality of sleeves, each of the sleeves including an open end and a closed end, an open end of each of the sleeves coupled proximate to a corresponding one of the apertures to enable each of the driver-fixator assemblies to rest against an interior portion of a corresponding aperture while a shaft of each of the driver-fixator assemblies is enclosed by one of the sleeves.

19. The system of claim 18 wherein the retainer portion includes a plurality of subsections, each of the subsections including indicia of a particular size fixation element so as to enable driver-fixator assemblies to be organized by the size of the fixation element.

20. The system of claim 19, wherein each of the subsections are removable to enable the driver-fixator assemblies in each subsection to be transported from one physical location to another physical location.

21. The system of claim 18, wherein the sleeves are tapered and each sleeve includes a length so that the closed end of each sleeve presses against each fixation element so as to prevent each fixation element from becoming uncoupled from the distal end of the shafts of the driver-fixator assemblies.

22. A system including:
a plurality of drivers, each of the drivers including a handle coupled to a shaft, the shaft including a distal tip opposite the handle that is configured to couple with a head of a surgical fixation element, each of the drivers including indicia of a particular length and a particular diameter; and
a plurality of surgical fixation elements, each of the plurality of surgical fixation elements being detachably coupled to the distal tip of a corresponding one of the drivers that has indicia of the length and diameter of the fixation element coupled to the driver.
